(19) United States
(12) Patent Application Publication
(10) Pub. No.: US 2005/0149001 A1
(43) Pub. Date: Jul. 7, 2005

(54) Operation support system and support method of operation support system

(75) Inventors: Akinobu Uchikubo, Iruma-shi (JP); Masakazu Gotanda, Tsukui-gun (JP); Takeaki Nakamura, Hino-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 10/961,987

(22) Filed: Oct. 8, 2004

(30) Foreign Application Priority Data
Oct. 9, 2003 (JP) 2003-351227

(51) Int. Cl. A61B 17/00; A61B 19/00

(52) U.S. Cl. 606/1; 128/897

(57) ABSTRACT

In an operation support system, an operation device portion and a support device portion are connected to each other through a communication line. The operation device portion comprises a controller which controls a plurality of medical devices and a signal transmitting device which transmits data to the support device portion side with a communication protocol to be transmitted to the communication line and receives data from the support device portion side.

This Application claim benefit of Japanese Patent Application No. 2003-351227 filed in Japan on Oct. 9, 2003, the contents of which are incorporated by this reference.

### BACKGROUND OF THE INVENTION

1. Field of the Invention

The present invention relates to an operation support system, in which an operation room is connected to a remote support room through a communication line and an operator in the operation room performs the operation while being supported from another operator in the support room, and a support method of the operation support system.

2. Description of the Related Art

Generally, an operator in an operation room directly performs the operation of a patient in an operation room. However, when the operator performs an inexperienced operation in the operation room, an operation support system is considered that the proper operation is possible for the patient in the operation room by supporting the operator in the operation room for the operation such as an instruction of an incised part from another operator at a remote position (remote support operator) who is well-experienced in the operation during the operation by connecting the operator in the operation room to the other remote support operator through a public line.

Japanese Unexamined Patent Application Publication No. 2000-270318 discloses, as one related art, a remote operation support system.

### SUMMARY OF THE INVENTION

According to the present invention, an operation support system in which an operation device portion and a support device portion are connected each other through a communication line, wherein the operation device portion comprises a controller which controls a plurality of medical devices and a signal transmitting device which transmits data to the support device portion side with a communication protocol to be transmitted to the communication line and receives data from the support device portion side, wherein the controller comprises a transmitting-data storing portion which stores predetermined data from control data of the plurality of medical devices as data to be transmitted, an address storing portion which stores an address on the communication line of the support device portion, and a data transmitting control portion which transmits the data to be transmitted to the support device portion through the communication line, and wherein the data transmitting control portion reads the address from the address storing portion upon monitoring the control data and finding change on the transmitting data, and transmits the transmitting data which is changed therein to the support device portion through the communication line.

Further, a support method of an operation support system for connecting a support device portion to an operation device portion through a communication line, comprising: a transmitting-data storing step of storing predetermined data to be transmitted from control data of the plurality of medical devices into a storing portion of a controller; an address storing step of storing an address on the communication line of the support device portion into the storing portion; and a data transmitting control step of reading the address from the storing portion upon monitoring the control data and changing the transmitting data, and transmits the transmitting data which is changed therein to the support device portion from the controller through the communication line.

### BRIEF DESCRIPTION OF THE DRAWINGS

FIG. 1 is a block diagram schematically showing the entire structure of an endoscope operation system according to a first embodiment of the present invention;

FIG. 2 is a diagram showing the detailed structure of the endoscope operation system shown in FIG. 1;

FIG. 3 is a block diagram showing the structure of a (first) system controller shown in FIG. 2;

FIG. 4 is a flowchart showing a processing flow of the (first) system controller shown in FIG. 3;

FIG. 5 is an explanatory diagram showing a data table stored in a RAM shown in FIG. 4; and

FIG. 6 is a diagram schematically showing the entire structure of an endoscope operation system shown in FIG. 1 according to one modification.

### DESCRIPTION OF THE PREFERRED EMBODIMENTS

Hereinbelow, a description is given of an embodiment of the present invention with reference to the drawings.

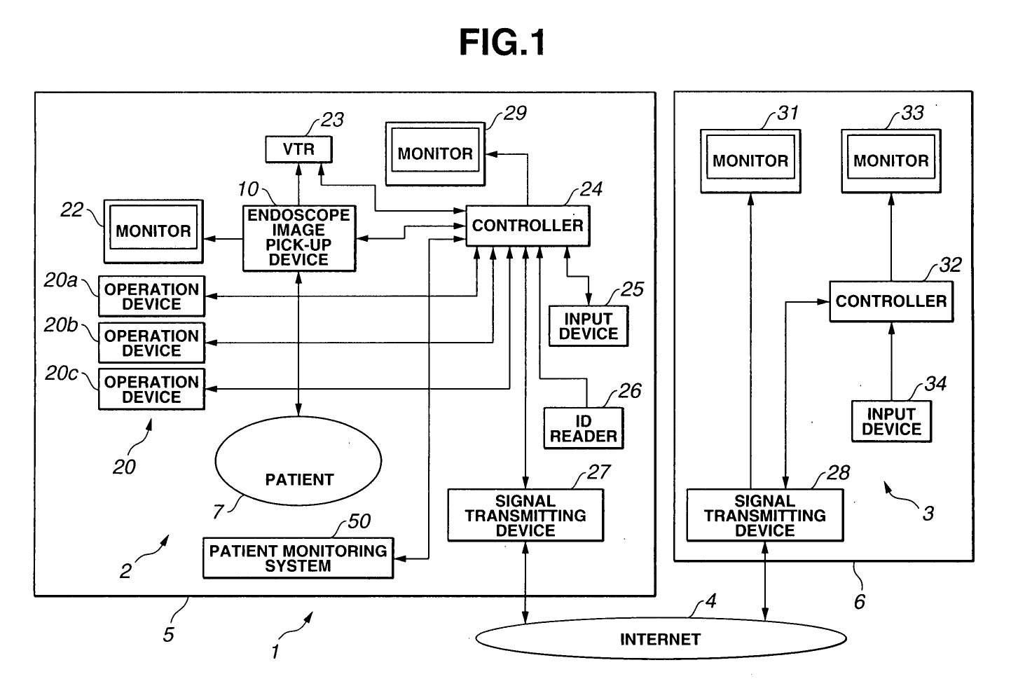

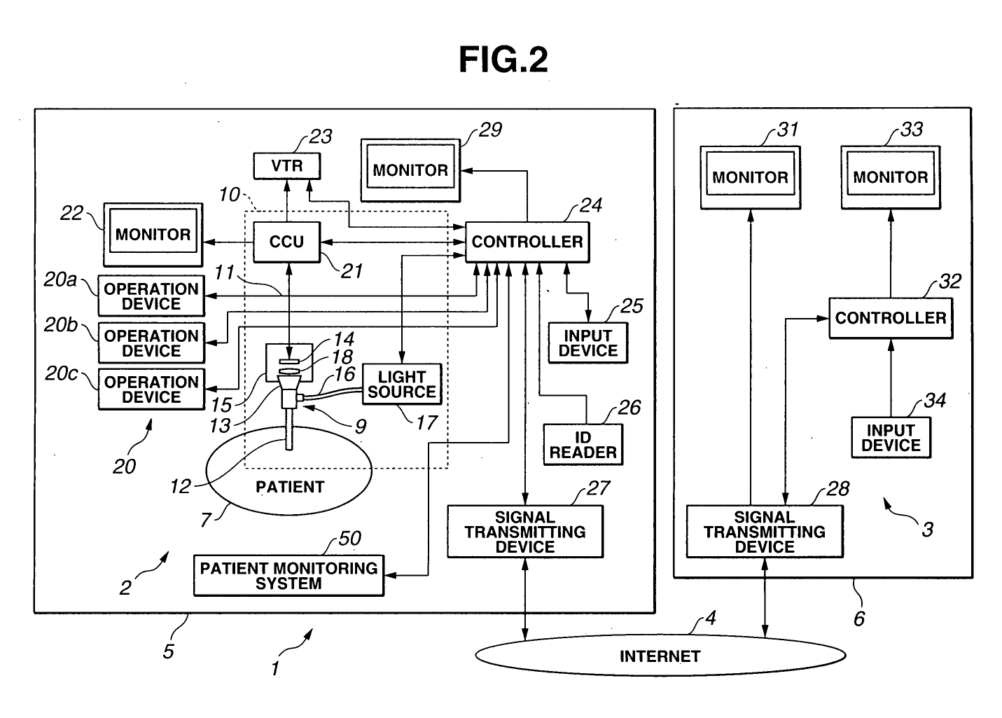

Referring to FIGS. 1 and 2, an endoscope operation system 1 according to the embodiment comprises: an endoscope operation device portion (hereinafter, abbreviated to an operation device portion) 2 for operation under endoscope observation; a support device portion 3 at a remote position of the operation device portion 2; an Internet communication line 4 through which the operation device portion 2 is connected to the support device portion 3; and an operation room 5 and a support room 6 to which the operation device portion 2 and the support device portion 3 are arranged, respectively.

Referring to FIGS. 1 and 2, for the purpose of a brief description, one operation room 5 and one support room 6 are connected to the Internet communication line 4. As will be easily understood, a plurality of operation rooms 5 and a plurality of support rooms 6 can be connected to the Internet communication line 4.

The operation device portion 2 arranged in the operation room 5 comprises: an endoscope image pick-up device 10 which observes the body cavity of a patient 7; a plurality of operation devices 20 which perform the operation of the therapeutic treatment of the patient 7 under the observation using the endoscope image pick-up device 10; and a patient monitoring device 50 which measures living-body information of the patient 7 (vital sign data).

Specifically, referring to FIG. 2, a treatment tool such as the operation device 20 for operation for therapeutic treatment and an optical endoscope 9 for observing the state of the operation using the treatment tool of the operation devices 20 are inserted in the abdominal cavity of the patient 7.

The operation device 20 comprises: a electric knife device 20a which performs the treatment such as incision or coagulation using an electric knife; an insufflator 20b which expands the abdominal cavity; and an ultrasonic treatment device 20c which crushes a stone or the like by ultrasonic waves.

The endoscope 9 is a rigid endoscope having a rigid inserting portion 12. A TV camera 15 including a charge-coupled device (hereinafter, abbreviated to a CCD) 14 is attached, as an image pick-up device, to an eyepiece portion 13 which is arranged to the rear end side of the inserting portion 12, and thus the endoscope 9 has means for picking-up an endoscope image.

A light guide cable 16 of the endoscope 9 is connected to a light source device 17, and transmits illumination light of a lamp (not shown) in the light source device 17 through a light guide in the light guide cable 16 and a light guide in the endoscope 9. Further, the light guide cable 16 outputs the transmitted illumination light from a light guide end surface fixed to an illumination window on a distal end side of the inserting portion 12, thereby illuminating an object such as the organ in the body cavity.

An observation window adjacent to the illumination window has an objective lens (not shown) for forming an optical image of the subject. The optical image is transmitted to the rear side by a relay lens system as optical image transmitting means arranged in the inserting portion 12, and is enlargedly observed through an eyepiece lens (not shown) of the eyepiece portion 13.

The optical image transmitted to the CCD 14 through an image forming lens 18 of the TV camera 15 which is detachably attached to the eyepiece portion 13 is formed. The CCD 14 is connected to a camera control unit (hereinafter, abbreviated to a CCU) 21 with a signal cable 19, and forms an endoscope image pick-up device 20 which performs the signal processing of a signal photoelectrically-converted by the CCD 14 to generate a standard video image.

The video signal is outputted to a first monitor 22 and a VTR 23 from the CCD 14, and the endoscope image of the organ in the body cavity and of the distal end side of a treatment tool of an operation device 20 picked-up by the CCD 14 is displayed. Further, the endoscope image is recorded to the VTR 23.

The CCU 21 and the operation device 20 are connected to a (first) controller 24 for controlling them. The data transmission/reception between the controller 24 and the light source device 17, the VTR 23, the CCU 21, and each operation device 20 is performed by connecting respective cables from the operation device 20 to a connector arranged to the controller 24 with a general serial communication protocol such as RS-232C or IEEE1394.

The controller 24 is connected to a touch panel 25 for inputting a control instruction and an ID reader 26 for reading data of an RF-ID chip or magnetic card for inputting patient data. The data is received/transmitted among the controller 24, the touch panel 25, and the ID reader 26 by a USB protocol.

The CCU 21 may control the changing operation of color tone through the controller 24 by operating the touch panel 25. Further, the power control of the operation device 20 may also be perfomed by operating the touch panel 25. In the case of using the electric knife device 20a as the operation device 20, a power level for incision or coagulation using the electric knife may be controlled. In the case of using the insufflator 20b as the operation device 20, the pressure set value may be controlled. In the case of using the ultrasonic treatment device 20c as the operation device 20, an ultrasonic output level may be controlled.

The ID reader 26 reads the patient data recorded to the RF-ID chip or magnetic card. The patient data is inputted to the controller 24, and the controller 24 outputs the patient data to the CCU 21. When the display of superimposing the patient data to the endoscope image is selected, the controller 24 controls the CCU 21 to superimpose the patient data to the endoscope image. This enables to display the endoscope image superimposed the patient data on the monitor 22. Further, the controller 24 reads living-body information of the patient 7 (vital sign data) from the patient monitoring device 50, and superimposes and displays the read living-body information (vital sign data) of the patient 7 to the endoscope image. The data transmission/reception between the controller 24 and the patient monitoring device 50 is performed by connecting the cable from the patient monitoring device 50 to the connector arranged to the controller 24 with the general serial communication protocol such as RS-232C.

The CCU 21 and the controller 24 arranged in the operation room 5 are connected to a signal transmitting device 27. The video signal of the endoscope image is outputted from the CCU 21 and is displayed on the monitor 22. The video signal of the endoscope image is outputted to the signal transmitting device 27 through the controller 24. Further, the signal transmitting device 27 may transmit video signal of the endoscope image to a signal transmitting device 28 on the support room 6 side through the Internet communication line 4 with the communication protocol to be transmitted on the Internet communication line 4. The communication protocol of the Internet communication line 4 is, e.g., TCP/IP, for communication through a dedicated line having a communication large-capacity (optical fiber line).

The controller 24 converts, into a video signal, a signal which is transmitted from the signal transmitting device 28 on the support room 6 side to the signal transmitting device 27 through the Internet communication line 4. Further, the controller 24 outputs the converted signal to a second monitor 29 connected to the signal transmitting device 27, and displays image information from the signal transmitting device 28 side on the second monitor 29.

A control signal or patient data from the controller 24 is converted into a signal to be transmitted from the signal transmitting device 27 through the Internet communication line 4. The data is transmitted to the signal transmitting device 28 on the support room 6 side through the Internet communication line 4.

A keyboard (not shown) or the like is connected to the controller 24. Character information is transmitted to the remote support operator on the signal transmitting device 28 side through the controller 24 from the keyboard. Although not shown, the controller 24 captures, as data, a voice signal from a voice input device for inputting the voice by a handsfree device attachable to the operator's head, by a voice recognizing function.

A third monitor 31 is connected to the signal transmitting device 28 in the support device portion 3 in the support room 6. The third monitor 31 displays the endoscope image transmitted from the CCU 21 on the operation room 5 side.

The signal transmitting device 28 is connected to a (second) controller 32. A fourth monitor 33 is connected to the controller 32. An input device 34 such as a touch panel or keyboard is connected to the controller 32. A pointing device such as a mouse (not shown) is connected to the controller 32.

The controller 32 captures, as a still image, the endoscope image transmitted from the signal transmitting devices 27 and 28 from the CCU 21 on the operation room 5 side, and receives the patient information transmitted through the signal transmitting devices 27 and 28 from the controller 24. Further, the controller 32 superimposes and displays the information on the fourth monitor 33. In order to provide support information such as instructing information upon operating for the operator in the operation room 5, the display of the position to be incised using the marking, the position of the artery to which the attention is paid upon, and so forth are inputted and displayed on the input device 34. The controller 32 overlays and displays the displayed and inputted image on the fourth monitor 33.

The image information overlaid and displayed on the fourth monitor 33 is transmitted to the second monitor 29 in the operation room 5 through the signal transmitting devices 27 and 28. The contents of the transmitted information are displayed on the display screen of the second monitor 29. The operator in the operation room 5 can perform the operation, watching the image displayed on the second monitor 29 which indicates the support information by the support operator in the support room 6, thereby the proper operation is accomplished.

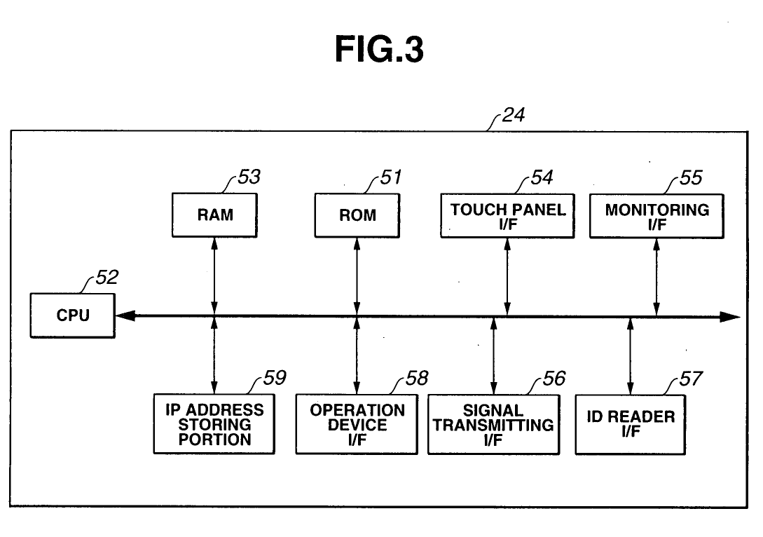

Referring to FIG. 3, the controller 24 comprises: a ROM 51 which stores a control program and the like; a CPU 52 as data transmitting control means which executes the control program in the ROM 51; a RAM 53 as transmitting data storing means which reads/writes various data used for the control operation of the CPU 52; a touch panel I/F portion 54 which receives/transmits the data from/to the touch panel 25; a monitoring I/F portion 55 which receives/transmits the data from/to the patient monitoring device 50; a signal transmitting I/F portion 56 which receives/transmits the data from/to the signal transmitting device 27; an ID reader I/F portion 57 which receives/transmits the data from/to the ID reader 26; an operation device I/F portion 58 which receives/transmits the data from/to the operation device 20; and an IP address storing portion 59 as address storing means which stores IP addresses of the signal transmitting devices 28 in the plurality of support room 6 as the destinations for transmitting the data through the signal transmitting device 27.

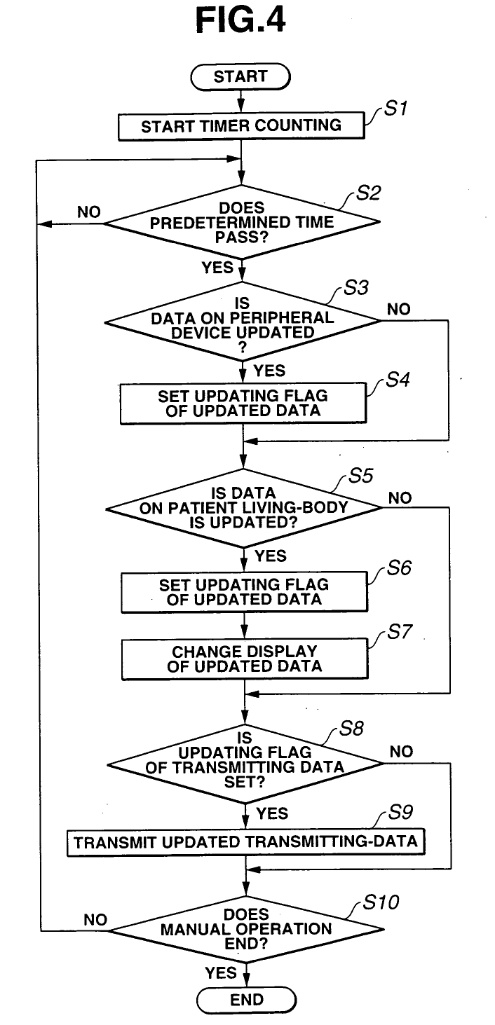

Hereinbelow, operations with the above-mentioned structure will be described according to the embodiment. Referring to FIG. 4, in step S1, the controller 24 starts the counting operation of an internal timer, thereby counting the time. In step S2, the controller 24 waits for a predetermined time. In step S3, the controller 24 determines whether or not the data of a peripheral device (operation device 20) is updated.

The controller 24 obtains, through the operation device I/F portion 58, various setting data and measurement data of the electric knife device 20a, the insufflator 20b, and the ultrasonic treatment device 20c used for the operation as the peripheral device (operation device 20) every predetermined time. Further, the controller 24 obtains the living-body information of the patient 7 (vital sign data) from the patient monitoring device 50 through the monitoring I/F portion 55.

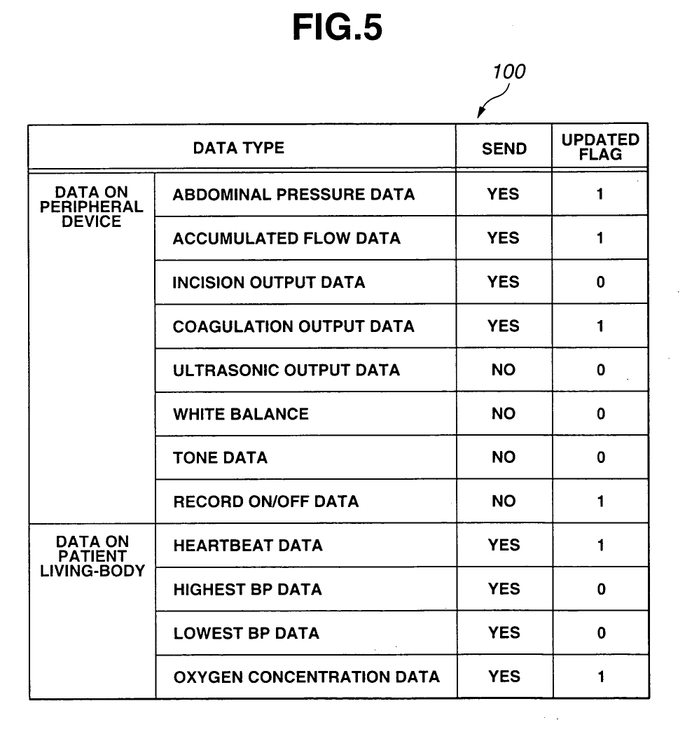

Referring to FIG. 5, the data on the living-body information (vital sign data) and the data on the peripheral device (operation device 20) are stored in the RAM 53 as table data 100, and are updated every predetermined time if changed. An updating flag is set to the data in the data group. If the data is changed, the updating flag is set (flag=1). By referring to the updating flag, the updating processing of data is executed.

In the table data 100, when one piece of data in the data group is updated, the data to be transmitted to the support room 6 is registered in advance as transmitting data.

Therefore, the controller 24 recognizes the updating state of the data every predetermined time by the updating flag, and performs the transmitting processing of the data indicating the updating flag 1 as the transmitting data to the support room 6.

Specifically, when it is determined that the data group on the peripheral device (operation device 20) is updated, in step S4, the controller 24 sets, to 1, the updating flag of the updated data in the data group of the peripheral device (operation device 20). Then, the processing advances to step S5. If it is determined in step S3 that the data group on the peripheral device (operation device 20) is not updated, the processing advances to step S5 without processing.

In step S5, it is determined whether or not data group in the living-body information (vital sign data) of the patient 7 is updated. If it is determined that the data group in the living-body information (vital sign information) is updated, in step S6, the updating flag of the updated data in the living-body information (vital sign data) is set to 1 and then the processing advances to step S7. If it is determined in step S5 that the data in the living-body information (vital sign data) is not updated, the processing advances to step S7.

In step S7, the display operation of the updated data is changed. In step S8, it is determined whether or not the data having the updating flag 1 exists in the transmitting data. Further, if it is determined that the data having the updating flag 1 exists, in step S9, the IP address of the signal transmitting device 28 in the support room 6 is read from the IP address storing portion 59, and the patient data obtained by the ID reader 26 from the transmitting data having the updating flag 1 is added to the IP address. The patient data additionally having the IP address is transmitted through the signal transmitting I/F portion 56. The processing subsequent to step S2 is repeated until the manual operation ends in step S10.

According to the embodiment, the controller 24 systematically manages the data group on the operation device arranged to the operating room and the data group in the living-body information (vital sign data). Further, upon changing the transmitting data which is registered in advance from the data group, only the transmitting data is transmitted to the support room 6 at the IP address which is stored in the IP address storing portion 59. The information necessary for the operation support for the support room 6 can be provided with the traffic of the Internet communication line 4 being maintained to be optimum. Thus, the support from the support room 6 is efficiently performed.

According to the embodiment, although the data to be transmitted is transmitted upon monitoring the updating state of the data group at the predetermined time interval by using the timer and updating the data, the present invention may not be limited to this. For example, upon generating an event for updating the data from the each operation device 20, the interrupt processing may be performed, the updating state may be monitored, and the data to be transmitted may be transmitted upon updating the data.

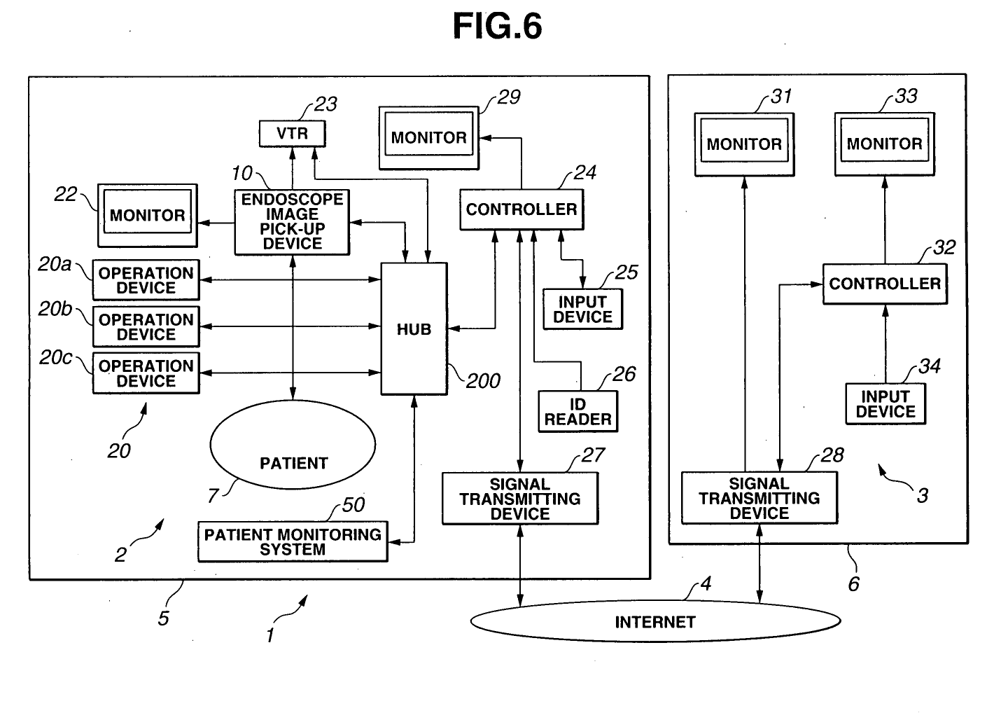

Further, according to the embodiment, as mentioned above, the patient monitoring device 50, the light source device 17, the VTR 23, the CCU 21, and the operation device 20 receive/transmit the data from/to the controller 24 through the general serial communication. However, the present invention may not be limited to this, and the patient monitoring device 50, the light source device 17, the VTR 23, the CCU 21, and the operation device 20 may be connected to the controller 24 through Ethernet®, thereby connecting the patient monitoring device 50, the CCU 21, and the operation device 20 to the controller 24 through a hub 200 as shown in FIG. 6 only by providing one Ethernet® connector to the controller 24, the data can be received/transmitted from/to the devices, and the connecting environment in the operation room 5 may be excessively improved.

As mentioned above, according to the embodiment, the support information can be received/transmitted in real time while the traffic of a data receiving/transmitting line is kept to be optimum. Further, the performance on data reception and transmission is improved.

Having described the preferred embodiment of the invention referring to the accompanying drawings. It should be understood that the present invention is not limited to those precise embodiments and various changes and modifications thereof could be made by one skilled in the art without departing from the spirit of scope of the invention as defined in the appended claims.

1. An operation support system in which an operation device portion and a support device portion are connected each other through a communication line,
wherein the operation device portion comprises a controller which controls a plurality of medical devices and a signal transmitting device which transmits data to the support device portion side with a communication protocol to be transmitted to the communication line and receives data from the support device portion side,
wherein the controller comprises
a transmitting-data storing portion which stores predetermined data from control data of the plurality of medical devices as data to be transmitted,
an address storing portion which stores an address on the communication line of the support device portion, and
a data transmitting control portion which transmits the data to be transmitted to the support device portion through the communication line, and
wherein the data transmitting control portion reads the address from the address storing portion upon monitoring the control data and finding change on the transmitting data, and transmits the transmitting data which is changed therein to the support device portion through the communication line.

2. The operation support system according to claim 1, wherein the operation device portion comprises a signal processing portion which generates an image signal for picking-up an image of an operation state of the medical device into a standard video signal, and the controller controls the signal processing portion.

3. The operation support system according to claim 1, wherein the operation device portion comprises a signal processing portion which generates an endoscope image signal for picking-up an image of an operation state of the medical device into a standard video signal, and the controller controls a light source device which supplies illumination light to the signal processing and an endoscope.

4. The operation support system according to claim 2, wherein the signal processing portion is connected to a monitor and an image recording portion, and outputs the signal-processed video signal to the monitor and the image recording portion.

5. The operation support system according to claim 3, wherein the signal processing portion is connected to a monitor and an image recording portion, and outputs the signal-processed video signal to the monitor and the image recording portion.

6. The operation support system according to claim 1, wherein the controller connects an instruction input portion for instructing the control operation to a patient data input portion for inputting patient data.

7. The operation support system according to claim 1, wherein the support device portion comprises:
a signal transmitting device which receives/transmits data from/to the signal transmitting portion of the operation device portion through the communication line;
a support-device-portion controller; and
an input device which is connected to the support-device-portion controller.

8. The operation support system according to claim 1, wherein the plurality of medical devices are connected to the support-device-portion controller by a common network protocol.

9. The operation support system according to claim 8, wherein the common network protocol is an Ethernet (registered trademark) protocol.

10. The operation support system according to claim 8, wherein a protocol of the communication line is an Internet protocol.

11. An operation support system in which an operation device portion and a support device portion are connected each other through a communication line,
wherein the operation device portion comprises a controller which controls a plurality of medical devices, a signal transmitting device which transmits data to the support device portion side with a communication protocol to be transmitted to the communication line and receives data from the support device portion side, and a signal processing portion which generates a standard video signal of an image of an operation state picked-up by the medical device,
wherein the controller comprises
a transmitting-data storing portion which stores predetermined data from control data of the plurality of medical devices as data to be transmitted,
an address storing portion which stores an address on the communication line of the support device portion, and
a data transmitting control portion which transmits the data to be transmitted to the support device portion through the communication line, and
the data transmitting control portion reading the address from the address storing portion upon monitoring the control data and finding change on the transmitting data, and transmitting the transmitting data which is changed therein to the support device portion through the communication line, and
wherein the support device portion comprises: a signal transmitting device which receives/transmits data from/to the signal transmitting portion of the operation device portion through the communication line; a support-device-portion controller; and an input device which is connected to the support-device-portion controller, a monitor for displaying the image transmitted from the operation device portion is connected to the signal transmitting device of the support device portion, the support-device-portion controller captures the image transmitted from the operation device portion as a still image, combines the support image inputted by the input device and displays the combined image on another monitor, and transmits information on the still image combined to the support image to the operation device portion side through the signal transmitting device.

12. The operation support system according to claim 11, wherein the signal processing portion of the operation device portion is connected to a monitor and an image recording portion, and a signal-processed video signal is outputted to the monitor and the image recording portion.

13. The operation support system according to claim 12, wherein the operation device portion outputs the information on the still image combined to the support image transmitted from the support device portion to another monitor through the signal transmitting device and the controller.

14. A support method of an operation support system for connecting a support device portion to an operation device portion through a communication line, comprising:
a transmitting-data storing step of storing predetermined data to be transmitted from control data of the plurality of medical devices into a storing portion of a controller;
an address storing step of storing an address on the communication line of the support device portion into the storing portion; and
a data transmitting control step of reading the address from the storing portion upon monitoring the control data and changing the transmitting data, and transmits the transmitting data which is changed therein to the support device portion from the controller through the communication line.
